(19) United States
(12) Patent Application Publication
(10) Pub. No.: US 2008/0261881 A1
(43) Pub. Date: Oct. 23, 2008

(54) Use of human chorionic gonadotropin (HCG) orally or for injection for the treatment of mood disorders and alcoholism

(75) Inventor: Daniel Oscar Belluscio, Capital Federal (AR)

Correspondence Address: BELLUSCIO, DANIEL OSCAR, ESTADOS UNIDOS 972 10 E, CAPITAL FEDERAL / BUENOS AIRES, 1101, AR

(73) Assignee: Daniel Oscar Belluscio, Capital Federal (AR)

(21) Appl. No.: 12/007,596

(22) Filed: Jan. 14, 2008

(30) Foreign Application Priority Data
Apr. 23, 2007 (AR) P20070101739

(51) Int. Cl. A61K 38/24; A61P 25/32; A61P 25/00

(52) U.S. Cl. 514/12

(57) ABSTRACT

A new use of Human Chorionic Gonadotropin (HCG) diluted as previously established for the treatment of mood disorders and alcoholism.

The substance Human Chorionic Gonadotropin (HCG) to be used as medical therapy for effective treatment of mood disorders as well as highly effective treatment of alcoholism.

### DETAILED DESCRIPTION OF INVENTION

This invention relates to “THE USE OF HUMAN CHORIONIC GONADOTROPIN (HCG) ORALLY OR FOR INJECTION FOR THE TREATMENT OF MOOD DISORDERS AND ALCOHOLISM.”

### SCOPE

The substance Human Chorionic Gonadotropin (HCG) to be used as medical therapy for effective treatment of mood disorders as well as highly effective treatment of alcoholism.

### PREVIOUS ART

### Current Technique

Human Chorionic Gonadotropin (HCG) was found and described for the first time in pregnant women's urine by Ascheim and Zondek, about 1927. It was later found that this substance is produced in human placenta. Since it was discovered in 1927, it was recommended for countless uses. At present, it is mostly prescribed for fertility problems and cryptorchidism (failure of both testicles to descend in children). HCG is currently supplied as a lyophilized substance for injection. Material is drawn from pregnant women's urine. It is available from several international pharmaceutical laboratories.

### PROBLEM TO BE SOLVED

About 1954 an English investigator published a paper containing his own experience with this substance in the treatment of obesity. The paper was welcomed and accepted by scientists generally until 1974-75, when the method became obsolete.

The method provided by the investigator abovementioned had several problems: it was for injection, caused immunity after treatments longer than six weeks, had some secondary effects, such as fluid retention, and so on.

### THIS INVENTION CAN SOLVE THE PROBLEM

This invention aims to demonstrate that an HCG preparation for oral administration or for injection used either as a simple dilution or coupled to albumin or a cyclodextrin can be therapeutically effective in the treatment of mood disorders including (but not limited to) neurosis, irritability, depressive states, borderline states, and so on.

HCG preparation as above described is also effective in the treatment of all types of alcoholism.

### ADVANTAGES

HCG oral preparation provides the same therapeutic effects as the psychotropics commonly used in the treatment of the disorders as described above, but does not have the same technical and pharmacologic problems as such drugs.

Moreover, it is an alternative to be considered in the cases of alcoholism since there is no effective treatment for this condition yet.

The preparation can be used for longer periods without secondary effects.

### DESCRIPTION OF INVENTION

The standard lyophilised preparation supplied by pharmaceutical laboratories is used for HCG preparations. Originally, HCG is supplied as a lyophilised powder containing 2,000 to 10,000 International Units (IU) of HCG per vial. IU concept stands for an agreement whereby each IU represents the quantity that is adequate to cause maturity of an egg in experimental animals.

For the purposes of this invention, HCG is dissolved in 1% physiological saline with or without addition of human albumin or different buffers, to be administered as an injection or orally, placing it under the tongue and maintaining it there for an easier absorption by the rich sublingual venous plexus.

Dilutions are prepared in such a way that each cubic centimeter of diluted HCG corresponds to a certain quantity expressed as IU.

Once solution has been prepared in sterile conditions, it can be stored in the refrigerator for periods of 4 to 7 days. This period of time can be extended (7-10 days) if the solution is stored under cold chain conditions.

Once the solution has been absorbed by the sublingual mucosa, a fraction of HCG is absorbed and carried into the circulation until it reaches the regulation centers of hypothalamic region, which contain appetite and satiation centers and fatty tissue metabolism.

### APPLICATION EXAMPLES

Oral administration is more advantageous than injections one since it is easier to administer and equally effective. Since treatment is innocuous, it can be used for several months without problems and with equally effective results.

### The Following Study was Conducted in Order to Validate 105 Obtained Clinical Results:

Seventy (70) women were screened (double blind study was conducted at site Gynecology Section). After signing the required consent, they were divided into two groups: Group A received saline alone, whereas Group B received two different concentrations of HCG. The study was designed based on double blind study methods: neither the volunteers nor the staff knew who received placebo and who belonged to the HCG-administered group.

The numbers assigned to each volunteer showed the type of substance (placebo or HCG) to be administered. The envelopes containing the codes were opened at the end of the study.

### Determinations

The following tests were carried out during the study:

A—Laboratory studies (Day 0), and after the study.
B—Irritability test during treatment, which was evaluated through a questionnaire to be completed by patients once a week, including Hamilton test for depression and questionnaire for mood disorder evaluation.

All evaluations were performed by the same observer throughout the treatment period in order to avoid observation differences due to different observers performing evaluations.

### Study Period

Study period was five weeks, at the end of which the envelope containing the codes for each patient was opened, and the data obtained were used for statistical studies (regression and variance studies).

### Data Analysis

The following studies were performed:

Data were entered in a database and compiled in ASCII format.

Frequency, media, standard deviation and standard error analyses were conducted. Variance, co-variance and multiple regression analyses were conducted.

### Results

Volunteers completed a questionnaire concerning their mood during treatment.

The following statistical differences between both Groups were found:

HCG-administered patients felt better during study period (p<0.03 on the third week of treatment, and p<0.01 by the fifth week of treatment.) They had better and deeper sleep periods (p<0.06 on the third week of treatment.) They showed greater acceptance of points of view that were different from their own (p<0.01 on the fifth week of treatment.)

They were less irritable (p<0.001 from the fourth week of treatment.) They got less upset every time things were not as expected (p<0.05.)

They were less willing to argue for trifles (p<0.05.)

They were less inclined to argue loudly (p<0.005 on the fourth week of treatment.)

### Results:

After four weeks' treatment 65% of the treated patients reported that they were in a better mood, less irritable, had longer and better sleep periods, had a tendency to avoid arguing for trifles, and their familiar relationships were more friendly.

On the other hand, the volunteers who had problems with excess of alcoholic drinks reported that they “did not feel like drinking”, even when they kept away from liquor and even when social pressure was usually very strong. Approximately 10% of the patients completely quit alcoholic drinks spontaneously during treatment.

### Conclusions:

Mood disorders are a very common pathology in society nowadays, and the several treatments recommended are not always properly used for moderate to severe secondary effects.

The use of HCG has demonstrated efficacy in the treatment of mood disorders without revealing undesirable effects, as well as the capacity to be administered for long periods.

On the other hand, alcoholism is a serious social health problem for which there are no available therapeutic solutions. Since oral HCG has no secondary effects, its administration for the treatment of chronic alcoholism is an excellent and innocuous therapeutic aid.

The advantages of this invention, which are briefly described above, should not be limited to such description but added with additional contributions from users and experts in the art, and will become more apparent and better understood through the images of the tests performed, which are schematically shown below without references to scales, in the attached image wherein:

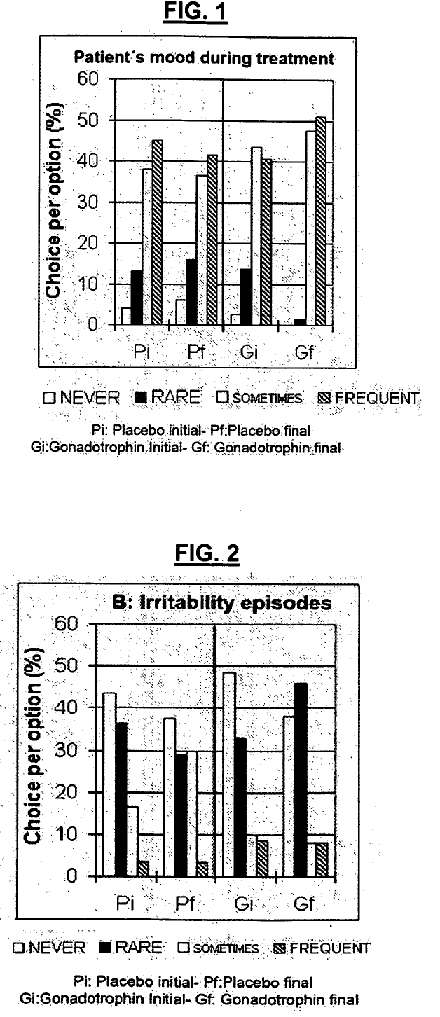

FIG. 1 is a chart of the tests performed showing patient's mood during treatment.

FIG. 2 is a chart of the tests performed showing irritability episodes.

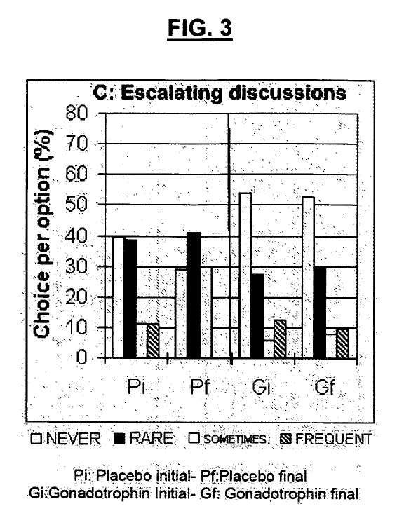

FIG. 3 is a chart of the tests performed showing the arguments held during treatment.

The advantages of this invention are plain from the description above as well as the images included, showing clear functional advantages of the product, characterizing the invention and representing a beneficial technological improvement that warrants the inclusion of the invention in the law with the pertinent legal protection as per the appended claims.

1. “THE USE OF HUMAN CHORIONIC GONADOTROPIN (HCG) ORALLY OR FOR INJECTION FOR THE TREATMENT OF MOOD DISORDERS AND ALCOHOLISM” characterized by HUMAN CHORIONIC GONADOTROPIN (HCG) being diluted in physiological saline, with or without the addition of human albumin, or a cyclodextrin or different buffers, in a proportion of 1%, to be administered either by injecting it or orally, as a sublingual form, maintaining it there for an easier absorption by the sublingual venous plexus.

2. “THE USE OF HUMAN CHORIONIC GONADOTROPIN (HCG) ORALLY OR FOR INJECTION FOR THE TREATMENT OF MOOD DISORDERS AND ALCOHOLISM” of claim 1, characterized by the solution being prepared in such a way that each cubic centimeter of diluted HCG corresponds to a certain quantity expressed as international units.

3. “THE USE OF HUMAN CHORIONIC GONADOTROPIN (HCG) ORALLY OR FOR INJECTION FOR THE TREATMENT OF MOOD DISORDERS AND ALCOHOLISM” of any of the previous claims, characterized by preparation of the solution under sterile conditions and at cold temperature, and future distribution under cold conditions.
